(19) United States
(12) Patent Application Publication
(10) Pub. No.: US 2026/0224778 A1
(43) Pub. Date: Aug. 6, 2026

(54) INJECTABLE BIOMATERIAL FOR MULTI-FUNCTIONAL PEPTIDE AND SMALL MOLECULE DRUG DELIVERY

(71) Applicant: ConductINK, LLC, Moorestown, NJ (US)

(72) Inventors: Louis Salvatore PAONE, Glassboro, NJ (US); Peter Adam GALIE, Moorestown, NJ (US)

(73) Assignee: ConductINK, LLC, Moorestown, NJ (US)

(21) Appl. No.: 19/324,536

(22) Filed: Sep. 10, 2025

Related U.S. Application Data
(60) Provisional application No. 63/706,821, filed on Oct. 14, 2024.

(51) Int. Cl. A61L 27/20; A61L 27/18; A61L 27/22; A61L 27/36; A61L 27/52; A61L 27/54; C08L 5/08

(52) U.S. Cl. CPC A61L 27/20; A61L 27/18; A61L 27/227; A61L 27/3687; A61L 27/52; A61L 27/54; C08L 5/08; A61L 2430/32

(57) ABSTRACT

An injectable carrier composition for drug delivery includes an amphiphilic tri-block copolymer surfactant in an amount from 10-25 wt. %; a non-sulfonated polysaccharide with at least 20% of subunit groups functionalized by 5-Norbornene 2-methylamine, present in an amount from 0.1-1.0 wt. %; a small molecule protein inhibitor in an amount from 0.1-1.0 wt. % or a large molecule antibody, present in an amount from 0.01-0.1 wt. %; and a peptide having a sequence containing either an amino or carboxy terminus GCG motifs, the peptide present in an amount from 0.1 to 1.0 wt. %. At room temperature, the carrier composition comprises a polymer network in solution, and upon injection to a subject having a temperature from 34-40° C., the polymer network of the carrier composition is configured to change to a hydrogel. Methods of making and methods of use are provided.

### RELATED APPLICATIONS

This application claims the benefit under 35 U.S.C. § 119 (e) of U.S. Provisional Patent Application No. 63/706,821 filed on Oct. 14, 2024, the contents of which are incorporated herein by reference in its entirety.

### SEQUENCE LISTING

The instant application contains a Sequence Listing which has been submitted electronically in XML file format and is hereby incorporated by reference in its entirety. Said XML copy, created on Mar. 25, 2026, is named CND00003USU1_SL.xml and is 3,680 bytes in size.

### TECHNICAL FIELD

The present disclosure relates generally to injectable compositions and more particularly to an injectable delivery system for small to large molecule therapeutics useful for repair or treatment of central nervous system injury and disease.

### BACKGROUND

Polysaccharides are long chains of carbohydrate molecules consisting of repeating sugar units called monosaccharides that play vital roles in the body. These complex sugars serve as structural components, energy reserves, and bioactive molecules. For example, in humans, glycogen—a polysaccharide—acts as a stored energy source, broken down into glucose when needed. Other polysaccharides, such as glycosaminoglycans in connective tissues, provide structural support. Additionally, polysaccharides are involved in cell signaling, immune responses, and regulating various biological processes.

Glycosaminoglycans (GAGs) or mucopolysaccharides are long, linear polysaccharides consisting of repeating disaccharide units (i.e., two-sugar units). The repeating disaccharide unit consists of a uronic sugar and an amino sugar, except in the case of the sulfated glycosaminoglycan keratan, where a galactose unit is in place of the uronic sugar. GAGs are found in vertebrates, invertebrates and bacteria. Being highly polar molecules that attract water, the body uses GAGs as lubricants or shock absorbers.

One type of GAG is hyaluronic acid (HA), also referred to as hyaluronan. Hyaluronic acid is a non-sulfonated, linear polysaccharide composed of repeating disaccharide units and has a very high molecular mass, ranging from 10⁵ to 10⁷ Da. HA can be synthesized by three transmembrane synthase proteins HAS1, HAS2, and HAS3, each of which is capable of transglycosylation when supplied with UDP-GlcA and UDP-GlcNAc. HAS2 is responsible for very large hyaluronic acid polymers, while smaller sizes of HA are synthesized by HAS1 and HAS3. While each HAS isoform catalyzes the same biosynthetic reaction, each HAS isoform is independently active. It is believed that through differences in enzyme activity and expression, the wide spectrum of biological functions mediated by HA can be regulated, such as its involvement with neural stem cell regulation in the subgranular zone of the brain.

Hyaluronic acid is a major component of synovial tissues and fluid, as well as the ground substance of other connective tissues. Hyaluronic acid binds cells together, lubricates joints, and helps maintain the shape of the eyeballs. The viscoelasticity of hyaluronic acid makes it ideal for lubricating joints and surfaces that move along each other, such as cartilage. A solution of hyaluronic acid under low shear stress has a much higher viscosity than while under high shear stress. Hyaluronidase, an enzyme produced by white blood cells, sperms cells, and some bacteria, breaks apart the hyaluronic acid, causing the solution to become more liquid.

In vivo, hyaluronic acid forms randomly kinked coils that entangle to form a hyaluronan network, slowing diffusion and forming a diffusion barrier that regulates transport of substances between cells. For example, hyaluronan helps partition plasma proteins between vascular and extravascular spaces, which affects solubility of macromolecules in the interstitium, changes chemical equilibria, and stabilizes the structure of collagen fibers.

Other functions include matrix interactions with hyaluronan binding proteins such as hyaluronectin, glial hyaluronan binding protein, brain enriched hyaluronan binding protein, collagen VI, TSG-6, and inter-alpha-trypsin inhibitor. Cell surface interactions involving hyaluronan are its well-known coupling with CD44, which may be related to tumor progression, and also with RHAMM (Hyaluronan-mediated motility receptor), which has been implicated in developmental processes, tumor metastasis, and pathological reparative processes.

Fibroblasts, mesothelial cells, and certain types of stem cells surround themselves in a pericellular “coat”, part of which is constructed from hyaluronan, in order to shield themselves from bacteria, red blood cells, or other matrix molecules. For example, in regard to stem cells, hyaluronan, along with chondroitin sulfate, helps to form the stem cell niche. Stem cells are protected from the effects of growth factors by a shield of hyaluronan and minimally sulfated chondroitin sulfate. During progenitor division, the daughter cell moves outside of this pericellular shield where it can then be influenced by growth factors to differentiate even further.

### SUMMARY

The present disclosure is directed to an injectable composition that provides a non-invasive strategy to promote CNS tissue regeneration and re-connectivity. The composition can inhibit the fibrotic response caused by infiltrating fibroblasts and can block a cofactor's receptor binding site that inhibits axon outgrowth to encourage neural regeneration.

According to some embodiments of the present disclosure, the composition utilizes a combinatorial approach of incorporating and exploiting norbornene-hyaluronic acid as a functional carrier of multiple therapeutic agents aimed at curbing fibrosis and stimulating host neuronal regeneration. The combination of large and small bioactive agents provides the end user tunability for an array of disease treatments. Embodiments of the present disclosure are particularly targeted to spinal cord injury by loading a neuronal growth peptide and small molecule inhibitor to halt fibrosis/gliosis; however, this strategy can be applied to other tissues and therapeutic applications.

A system according to the present disclosure provides a multifunctional bioactive domain delivery system within a thermally responsive hydrogel scaffold for non-invasive treatment of CNS injury and disease. The specific embodiments defined in this application utilize both a small peptide and small molecule TGF-beta inhibitor to prevent downstream gliosis and fibrotic scarring of injured spinal cord tissue. The P01 peptide is also an inhibitor of the repulsive guidance molecule A cofactor that contributes to stimulating regeneration of host neuronal cells into the injured tissue.

The features and advantages described herein are not all-inclusive and, in particular, many additional features and advantages will be apparent to one of ordinary skill in the art in view of the drawings, specification, and claims. Moreover, it should be noted that the language used in the specification has been selected principally for readability and instructional purposes and not to limit the scope of the disclosed subject matter.

### BRIEF DESCRIPTION OF THE DRAWINGS

FIG. 1 illustrates a flow chart with processes in a method of preparing a carrier composition, in accordance with embodiments of the present disclosure.

FIGS. 2A-2C illustrate two reaction schemas depicting chemical processes that feature bioconjugations of a hyaluronic acid nanocarrier that result in “ink1”, “ink2”, or “ink3”, in accordance with embodiments of the present disclosure. The reaction schemas start in FIG. 2A, proceed with parallel processes in FIG. 2B, and proceed to ink1, ink2, or ink3 as shown in FIG. 2C.

FIGS. 3A-3D illustrate the combination of carriers with pluronic f127 triblock copolymer hydrogel to yield an injectable biomaterial for central nervous system injury in a spinal cord hemisection injury model as a method of use, in accordance with another embodiment of the present disclosure.

FIG. 4 provides a plot of a chemical characterization process called proton nuclear magnetic resonance to show successful conjugation of 5-Norbornene 2-methylamine to 60 kDa sodium hyaluronate to produce a hydrogel carrier, in accordance with an embodiment of the present disclosure.

FIG. 5 contains overlaid FTIR spectra of norbornene-hyaluronic acid with and without bioconjugation of TGF-β inhibitor, SB431542, which is identifiable by the peak at 1451 cm⁻¹ due to the absorption of infrared light at that wavenumber by its aromatic ring.

FIG. 6 provides UV-VIS spectroscopy of unmodified norbornene-hyaluronic acid, with a peak at 210-220 nm and norbornene-hyaluronan bioconjugated with TGF-beta small molecule inhibitor, SB431542, identified by the presence of an absorbance shoulder at 300-350 nm.

FIG. 7 provides UV-VIS spectroscopy verifying bioconjugation of RGMa antagonist peptide (P01), GCGGRQVGRYLTFA, to norbornene-hyaluronic acid using the absorption of two aromatic amino acids present in the sequence that absorb light (Phenylalanine at 250 nm, Tyrosine at 280 nm), in accordance with embodiments of the present disclosure. The absorbance of unmodified norbornene-hyaluronan is also shown.

FIG. 8 depicts UV-VIS spectroscopy of Alexa Fluor 633-Immunoglobulin G successfully conjugated to norbornene-hyaluronic acid using the characteristic absorbance for IgG @ 280 nm along with absorbance of the Alexa Fluor. Unmodified norbornene-hyaluronic acid is again identifiable by its peak at 210-220 nm.

FIG. 9 provides overlaid UV-VIS spectra of the final products of chemical processes for ink1 and ink2, in accordance with embodiments of the present disclosure.

FIG. 10 contains a scanning electron micrograph of 20 weight percent pluronic f127.

FIG. 11 provides scanning electron micrographs showing the morphology of several carriers engineered with different chemical entities. Some of these include, but are not limited to, different intermediary steps within the chemical processes in the method, in accordance with embodiments of the present disclosure.

FIG. 12 shows a plot of release rates versus time for different carrier compositions from pluronic f127 in vitro.

FIG. 13 contains fluorescence microscopy images of normal human dermal fibroblasts seeded within a collagen hydrogel. The hydrogel features two parallel 180-micron diameter channels: ink1 was combined with pf127 and injected into one channel, and unmodified norbornene-hyaluronan was combined with pf127 and injected into the adjacent channel. The hydrogel was incubated for three days at 37° C., fixed and stained for α-smooth muscle actin, a marker for fibroblast activation. Fibroblasts adjacent to the channel receiving ink1 demonstrated significantly lower levels of α-smooth muscle actin-positive staining.

FIG. 14A depicts the modularity and tunability of using norbornene-hyaluronic acid as a bioactive nanocarrier for the combinatorial delivery of small molecules, peptides, and/or antibodies. FIG. 14A also schematically illustrates a method of use as an injectable, thermally responsive biomaterial to repair central nervous system injury, in accordance with an embodiment.

FIG. 14B illustrates an example of the method of use as an injectable, thermally responsive biomaterial to repair central nervous system injury, specifically a cervical hemisection injury model in Sprague-Dawley rats, which is shown in a photograph from an animal surgery.

FIG. 15 provides fluorescence microscopy images of histological slices of a rat spinal cord at two weeks post injury. Several different configurations of modified norbornene-hyaluronic acid were combined with pf127 and injected into a cervical hemisection injury model in Sprague-Dawley rats. Increased levels of Tuj (marker for beta-tubulin III) indicate the presence of host axons infiltrating into the injured tissue. GFAP or glial fibrillar acidic protein is a common marker for activated astrocytes. Therefore, positive staining in the injury area reveals astrocyte infiltration. Animals receiving ink1 combined with pf127 demonstrate the highest levels of axon (Tuj) and astrocyte (GFAP) infiltration.

FIGS. 16A-16D show fluorescence microscopy images of histological slices of the rat spinal cord at eight weeks following injury, where animals received pf127 containing unmodified norbornene-hyaluronan (“blank”) ink1 (“treatment”). Staining for axon and astrocyte markers Tuj and GFAP again revealed animals in the treatment group had higher levels of these markers in the injury area.

FIGS. 17A and 17B show fluorescence microscopy images of axon tracing, an additional way to evaluate the efficacy of biomaterials to repair the injured spinal cord. Both reticularmotor (BDA) and rubrospinal (GFP) tracts were traced by injecting these tracers into the brain and imaging the injury area at eight-weeks. Again, blank (unmodified norbornene-hyaluronan) and treatment (ink1) conditions were combined with pf127 were compared. Only the treatment condition resulted in successful connectivity across the injury site.

The figures depict various embodiments of the present disclosure for purposes of illustration only. Numerous variations, configurations, and other embodiments will be apparent from the following detailed discussion.

### DETAILED DESCRIPTION

Disclosed are an injectable delivery system for restoration of functional CNS tissue following initial insult, methods of use, and methods of making the same. In accordance with some embodiments, the system can be utilized as part of a regenerative medicine approach to initiate healing and regeneration of damaged neurological tissue. Some uses include treating or repairing CNS injury and disease as well as wound healing applications. For example, the injectable delivery system can be used to restore functionality to injured tissue and regenerate new tissue through inhibition of fibrotic processes.

Traditional injectable therapeutic modalities are limited to single chemical entities released by simple diffusion or enzymatic degradation through a hydrogel carrier system. Thus, a need exists for an improved drug delivery system. The present disclosure addresses this need by providing an injectable drug delivery system that leverages norbornene-hyaluronic acid as a combinatorial multi-faceted approach to deliver both chemical and biological entities on the same carrier for a more targeted and desirable outcome to dictate cell functionality.

### Example Embodiments

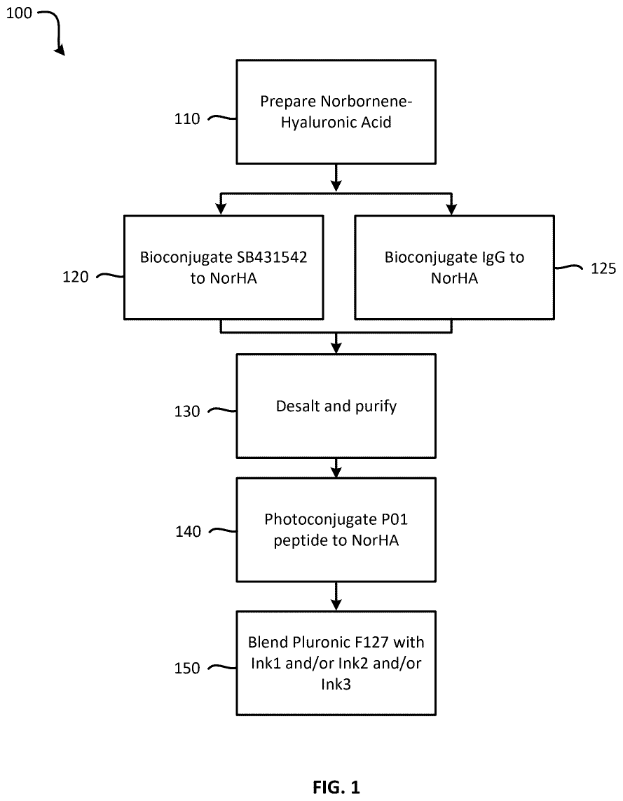

FIG. 1 illustrates a flow chart showing processes in a method 100 of making a carrier composition based on norbornene-hyaluronic acid (NorHA), in accordance with some embodiments.

Process 120 includes mixing norbornene-hyaluronic acid (NorHA) (produced via method 110) with a small-molecule drug, such as SB431542, an inhibitor of TGF-β signaling. NorHA is a non-sulfonated glucosaminoglycan that has been chemically modified at ratios of 0.2-1.0 with 5-Norbornene 2-methylamine. SB431542 and NorHA are stirred at 4° C. for 24 hours in the presence of N-Hydroxysuccinimide (NHS), 1-Ethyl-3-(3-dimethylaminopropyl) carbodiimide (EDC), and dimethyl sulfoxide (DMSO). To the resulting mixture is added a cysteine-containing bioactive peptide, via photoconjugation in process 140, which yields “ink1”.

Process 125 includes mixing NorHA with a large molecule drug, such as an immunoglobulin (IgG) molecule. NorHA is mixed with 0.5 M Sulfo-NHS in MES buffer prior to addition of EDC at a 15× molar ratio over Sulfo-NHS. IgG is added to the reaction for 5 hours at 22° C. To the resulting mixture is added a cysteine-containing bioactive peptide, again via photoconjugation in step 140, which yields “ink2”.

In one embodiment, steps 120 and 125 can be combined to conjugate both small and large molecule drugs to NorHA, prior to photoconjugation using step 140 to add a cysteine-containing bioactive peptide, yielding “ink3”.

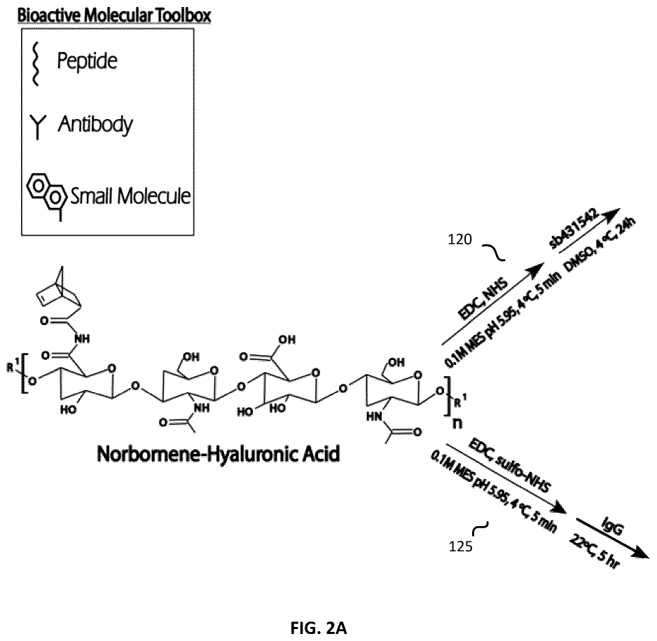

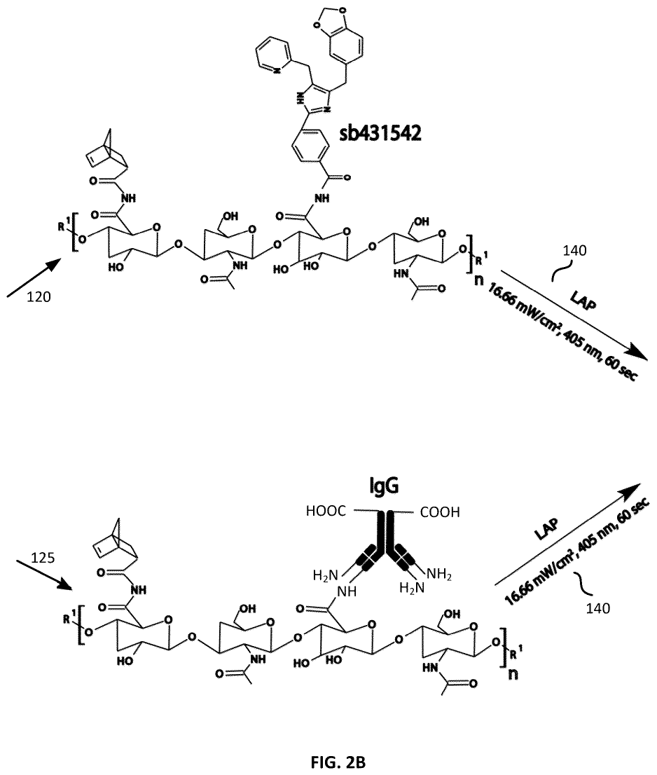

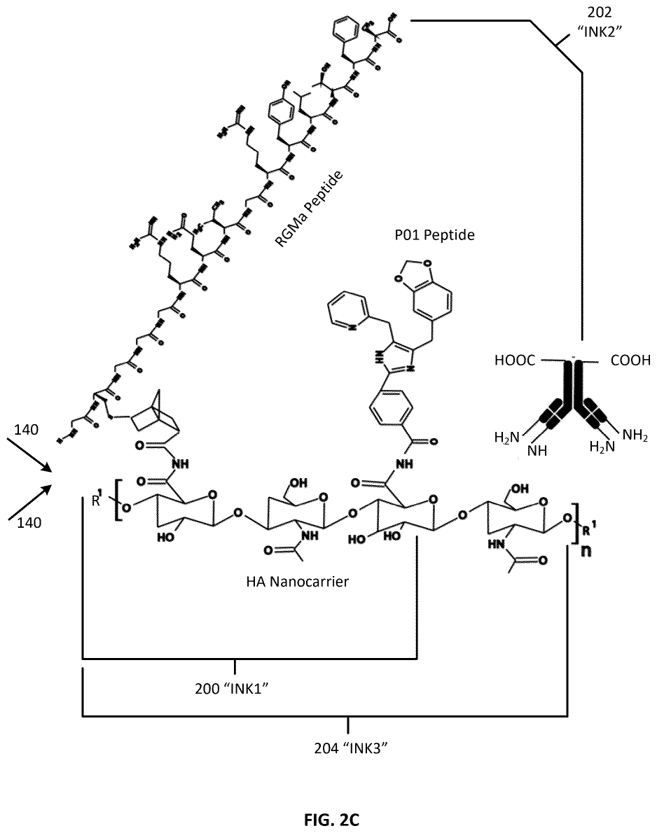

FIG. 2 illustrates two, distinct chemical reaction schemes for producing a first carrier composition that is suitable with small molecule drugs via method step 120 and large molecule drugs via method step 125. FIG. 2 uses SB431542 and an IgG molecule as examples of small and large drugs, respectively, in accordance with an embodiment. Method steps 120 and 125 can be performed separately to create a carrier of either small or large drugs or in series to combine both small and large drugs in a single carrier. FIG. 2 also illustrates a chemical reaction for making a second carrier composition that incorporates bioactive peptides using method step 140, in accordance with an embodiment.

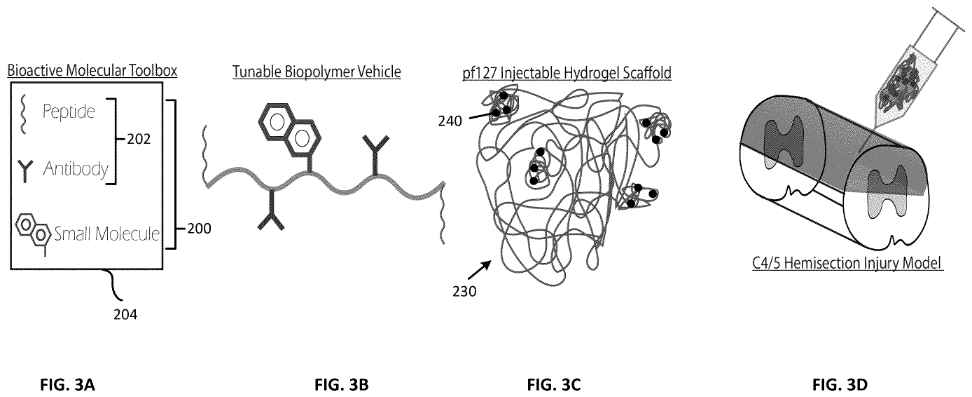

FIGS. 3A-3D illustrate a hydrogel carrier with peptide or drug carrier molecules, hydrophobic domains, and hydrophilic domains that can be injected into the site of a central nervous system injury, in accordance with embodiments of the present disclosure.

### Synthesis of Norbornene-Hyaluronic Acid

Referring to FIG. 1, method 100 begins with providing or preparing 110 a non-sulfonated polysaccharide that has been chemically modified (e.g., functionalized) with 5-Norbornene 2-methylamine. In some embodiments, the non-sulfonated polysaccharide is or includes glucosaminoclycan. In some embodiments, at least 20% of the of the non-sulfonated glucosaminoglycan (e.g., hyaluronic acid) are functionalized. In some embodiments, the non-sulfonated glucosaminoglycan is hyaluronic acid and is modified with norbornene to result in norbornene-hyaluronic acid (NorHA). In one embodiment, process 110 is performed as discussed below.

In a round bottom flask, 2-5 wt. % sodium hyaluronate is dissolved in deionized water for about 20 minutes to provide an aqueous HA solution. For example, 2 g of Na-HA is dissolved in 57 mL of DI H₂0. A fine mesh spherical ion exchange resin, such as Dowex 50 W×200, is added to the HA solution at a 3:1 mass ratio (e.g., 6 grams of Dowex to 2 grams of Na-HA) and stirred for about 2 hours, resulting in a HA-resin mixture. The HA-resin mixture is then filtered. In one embodiment, filtering is performed with vacuum filtration through cellulose filter paper, such as #2 Whatman filter paper or equivalent. Tert-butyl alcohol (TBA) is diluted 50% with water (1:1 ratio with H₂O), yielding about 30 mL of dilute TBA. The filtered HA resin mixture is added to the TBA and neutralized to a pH ~7.02-7.05 in a beaker. In one example, 21 mL used to achieve pH of 7.03. After neutralizing, the solution is partitioned into 50 mL tubes, frozen, and then lyophilized. Acid catalyzed dehydration is exploited using tert-butyl alcohol as a bridge for primary amine substitution of 5-Norbornene 2-methylamine at the primary alcohol subunit domain of the polymer. 100:1 addition of 5-Norbornene 2-methylamine (2 millimoles) per 20 nanomoles of hyaluronic acid is used to yield a chemical modification of 20 to 100 percent of a total of 158 sites per polymer based on a 60 kDa molecular weight. In some embodiments, the modified hyaluronic acid has a molecular weight from 10 kDa to 2 MDa.

At 65° C., two round bottom flasks with a magnetic stirrer and cannula are placed in an oven for about 10 minutes. 2 g of HA-TBA is manually torn into smaller fragments. A needle attached to a tube is connected to nitrogen flow. A first flask is capped with a stopper and the flask is allowed to cool. The stopper is removed, followed by adding HA-TBA and closing the flask again. The first flask is fully capped at two of the three openings and 260.785 μL of Nor-Amide is added, followed by capping the last (third) opening of the flask. One side of the flask is attached to a lab ring stand and placed under a magnetic stirrer at a stirring rate at or below 350 RPM. The cannula is removed from the oven and the HA-tert-butyl alcohol and Nor-Amide are mixed in the same flask. The cannula is placed in anhydrous dimethyl sulfoxide (DMSO) and connected to the cap of the flask. The nitrogen tube and needle are placed in the DMSO and 101.4 mL is perfused into the reaction drop-wise. The nitrogen needle is then removed from the DMSO then the cannula is removed from both sides. The solution is allowed to mix.

In a second flask, 901.562 mg of Benzotriazole-1-yl-oxy-tris-(dimethylamino)-phosphoniumhexafluorophosphate (“also known as Castro's Reagent”) (“BOP”) is added to 80.92 mL of DMSO and the 3 neck openings of the second flask are fully closed. The cannula is again placed in anhydrous DMSO and connected to the cap of the second flask. The nitrogen tube and needle are placed in DMSO and 101.4 mL is perfused into the reaction drop-wise. The nitrogen needle is removed from DMSO then the cannula is removed from both sides. The BOP is allowed to mix.

The nitrogen tube is placed on the second flask containing the BOP, the middle cap is vented, and the cannula is placed on the right side opening to connect to the first flask, which contains the Nor-Amide and HA-TBA. The second flask is vented with Nor-Amide and HA-TBA. The second flask containing the BOP is stirred at a higher stirring rate above 350 RPM.

After about 1 hour 40 minutes, a conical tube is filled with pure water and placed in a beaker with ice. The stopper is opened, and 10 mL of cold pure H₂O is injected into the second flask. A dialysis tube (e.g., 6-8 kDa) is placed in pure water and the end of the dialysis tube is clasped after opening and injecting all the NorHA solution into the tube. Then, 5 g of sodium chloride (NaCl) is added to 4 L water in a beaker. The water is changed twice a day for three days, adding 5 g of NaCl every time. Following that, the water is changed every day for and two days without adding NaCl.

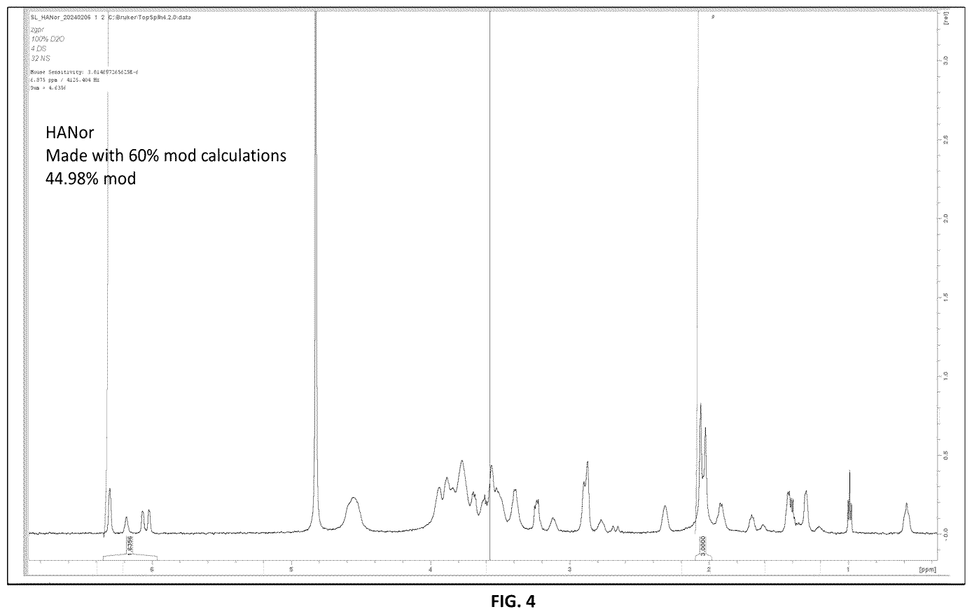

The NorHA solution is vacuum filtered and the NorHA solution is transferred into new dialysis tubing (6-8 kDa). For example, new dialysis tubing (6-8 kDa) is placed in pure water and the end of the dialysis tubing is clasped after opening and injecting all the NorHA solution into the tube. The water is then changed twice daily for three days, followed by transferring all the content to a 50 mL conical tube and stored overnight at −80° C. The contents are lyophilized for five days then purged with nitrogen gas (N₂) before storing at −20° C. The synthesized NorHA macromer had ~45% of its repeat units functionalized with norbornene, as analyzed with ¹H NMR spectroscopy (Shown in FIG. 4). The percentage of modification was calculated by comparing the integral of the methyl HA peaks between δ 1.9-2.1 ppm to the vinyl proton peaks of norbornene between δ 6.0-6.3 ppm.

### Bioconjugation of Sb431542 to Norbornene-Hyaluronic Acid

Method 100 continues with process 120 of bioconjugating SB431542 or other small molecule therapeutics (e.g., serotonin) to NorHA. Process 120 is depicted in the top portion of FIG. 2A. Process 120 includes preparing a solution with 1 mg/mL of Norbornene-Hyaluronic Acid (NorHA) in pH 5.95 2-(N-morpholino)-ethanesulfonic acid buffer (MES) and sonicated for about 5 minutes. 100 μL of 0.5 M N-Hydroxysuccinimide (NHS) (e.g., 0.05 millimoles of NHS) in pH 5.95 0.1 M MES buffer is added to the NorHA solution. 1-Ethyl-3-(3-dimethylaminopropyl) carbodiimide (EDC) (e.g., 0.74941 millimoles) is added into the reaction to provide a 15× molar ratio over NHS. 50 mM SB431542 is prepared in ultra-pure DMSO and added into the reaction. The reaction is incubated at 4° C. for 24 hours with gentle mixing.

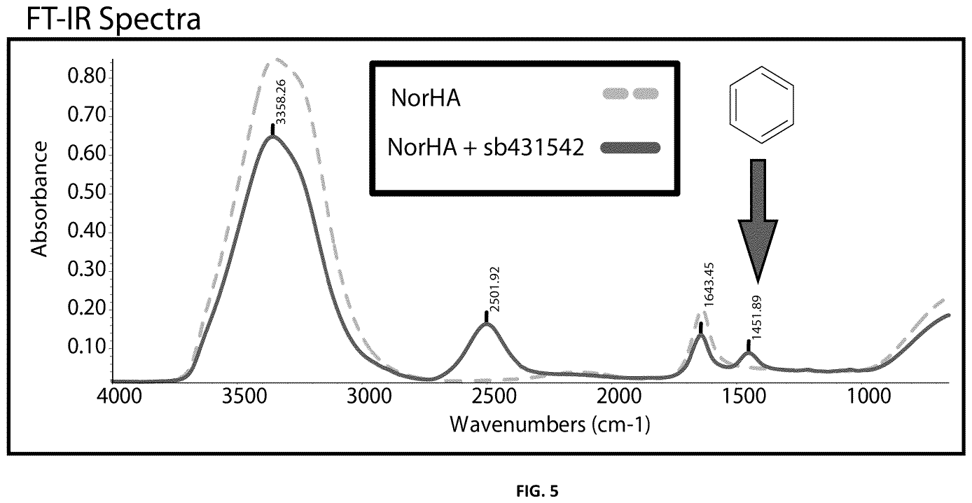

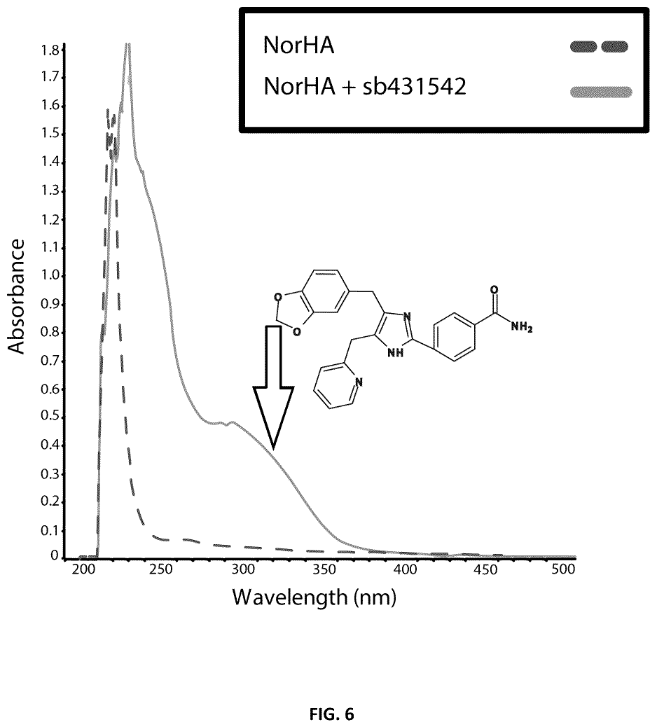

Process 120 proceeds with desalting and purifying 130 the sample. In one embodiment, process 130 is performed using 40K Zeba Desalting Columns. For example, process 130 includes removing the storage buffer by centrifuging at 700×g for 2 minutes at 15° C., adding 2 mL of phosphate-buffered saline (PBS) and centrifuging at 700×g for 2 min at 15° C., adding 2 mL of PBS and centrifuging at 700×g for 3 min at 15° C., adding 2 mL of PBS and centrifuging at 700×g for 4 min at 15° C., and terminating the reaction by removing unbound small molecule (e.g., SB431542) through size exclusion chromatography and centrifuging at 700×g for 3 min at 15° C. to collect the desalted and purified sample in PBS. The sample is then concentrated with a 10 k molecular weight cut-off concentrator (MWCO) by adding 1 mL of 7.4 pH PBS to the sample and centrifuging in column at 7,500×g for 20 min at 10° C. The final product was verified by Fourier Transform Infrared Spectroscopy (FTIR) picking up aromatic hydrogen stretching on SB431542 at 1451 cm⁻¹ (Shown in FIG. 5). Additionally, UV-VIS spectroscopy was performed to further support successful modification which shows a shoulder between 300-350 nm (SB431542 absorbance) paired with a sharp peak at 210-220 nm (NorHA) (Shown in FIG. 6).

### Bioconjugation of Igg to Norbornene-Hyaluronic Acid

In addition, or in the alternative, method 100 continues with process 125 of bioconjugating Immunoglobulin G (IgG) or other large molecules (e.g., neurotrophin 3 (NT3) or brain-derived neurotrophic factor (BDNF)) to NorHA. In some embodiments, the immunoglobulin G is a monoclonal antibody raised against an inflammatory cytokine, an axonal repulsive guidance cue, or another ligand associated with secondary injury. Process 125 is depicted in the bottom portion of FIG. 2A. Process 125 includes preparing a solution with 1 mg/mL of NorHA in pH 5.95 MES buffer and sonicated for 5 minutes. Next, 100 μL of 0.5 M Sulfo-NHS (e.g., 0.05 millimoles) in pH 5.95 0.1M MES Buffer is prepared and added into the NorHA solution. EDC is then added to the reaction for a 15× molar ratio over Sulfo-NHS (e.g., added to 0.74941 millimoles Sulfo-NHS) and incubated for 5 minutes at room temperature. 50 mM SB431542 is prepared in ultra-pure DMSO and added to the reaction. 500 μL of 7.5 wt % of Sodium Bicarbonate (NaHCO₃) with a pH of 8.14 is added, raising the pH to 7.4. Then, 150 μL of 2 mg/mL IgG is added, vortexed, and incubated for 5 hours at 4° C. under gentle mixing.

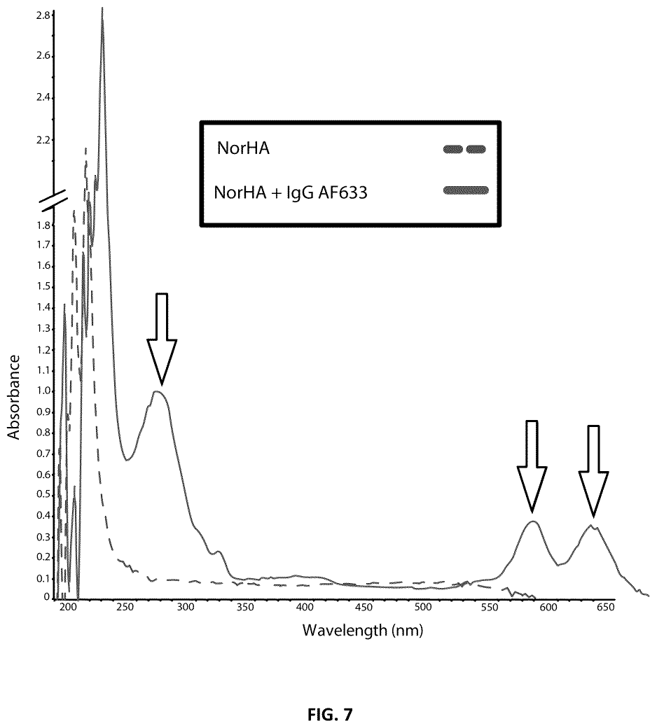

Process 125 continues with desalting and purifying 130 the sample. As noted above, process 130 can be performed with 40K Zeba desalting columns. In one embodiment, desalting and purification includes removing the storage buffer by centrifuging at 700×g for 2 minutes at 15° C., adding 2 mL of PBS and centrifuging at 700×g for 2 minutes at 15° C., adding 2 mL of PBS and centrifuging at 700×g for 3 minutes at 15° C., adding 2 mL of PBS and centrifuge at 700×g for 4 minutes at 15° C., and terminating the reaction by removing unbound large molecule (e.g., IgG) through size exclusion chromatography and centrifuging at 700×g for 3 minutes at 15° C. The desalted and purified sample is collected in phosphate-buffered saline (PBS). Next, the sample is concentrated using a 10 k MWCO concentrator, adding 1 mL of 7.4 pH PBS to the sample, and centrifuging in a column at 7,500×g for 20 minutes at 10° C. The final product was verified by UV-VIS spectroscopy to further support successful modification which shows a peak at 280 nm (IgG absorbance) paired with a sharp peak at 210-220 nm (NorHA) (Shown in FIG. 7).

### Photoconjugation of P01 Peptide to Norbornene-Hyaluronic Acid

Method 100 proceeds with photoconjugating 140 P01 peptide to NorHA~IgG, NorHA~SB431542, or NorHA~IgG~SB431542. Process 140 is illustrated FIG. 2B. In one embodiment, such as shown in the upper part of FIG. 2B, process 140 includes preparing a photoconjugation reaction with 34 mM Lithium phenyl-2,4,6-trimethylbenzoylphosphinate (LAP), 30 μL of 10 mM P01 peptide, and 10 μL of 10 mg/mL SB431542~NorHA. Alternatively, also as shown in the lower part of FIG. 2B for photoconjugating IgG~NorHA, process 140 includes adding 29 μL of 3.44 mg/ml IgG~NorHA construct to 34 mM LAP and 30 μL of 10 mM P01 peptide.

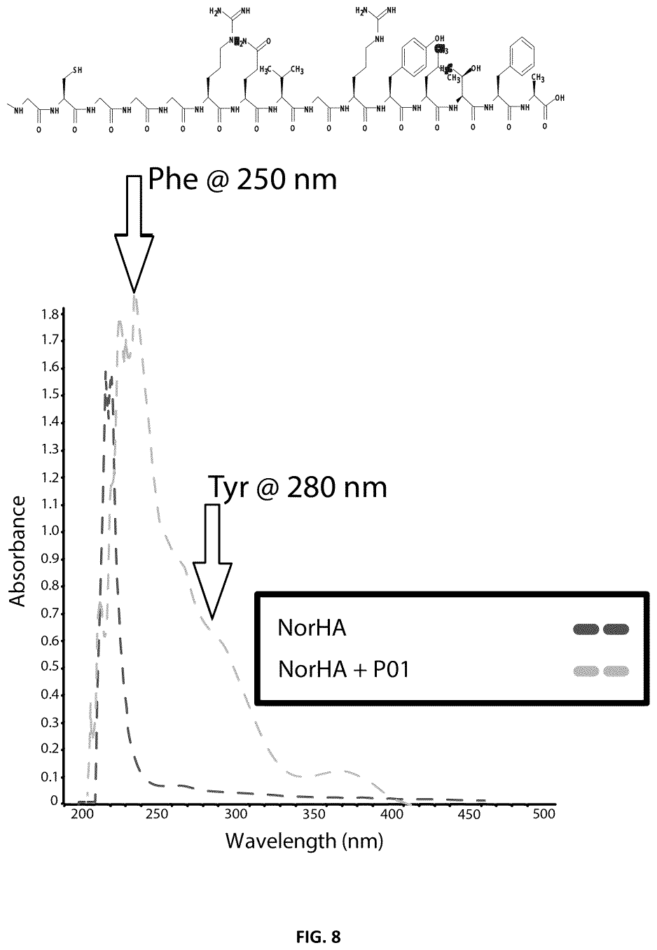

In either case, the resulting solution is vortexed and radiated with 405 nm light for about 60 seconds. The solution is then added to 1 mL of PBS pH 7.4 and centrifuged in 10K MWCO at 7,500×g for 20 minutes at 10° C. The resulting mixture is stored at 4° C. The photoconjugation of P01 peptide to NorHA was verified by UV-VIS spectroscopy to further support successful modification which shows a phenylalanine peak at 250 nm and a tyrosine peak at 280 nm paired with a sharp peak at 210-220 nm (NorHA) (Shown in FIG. 8).

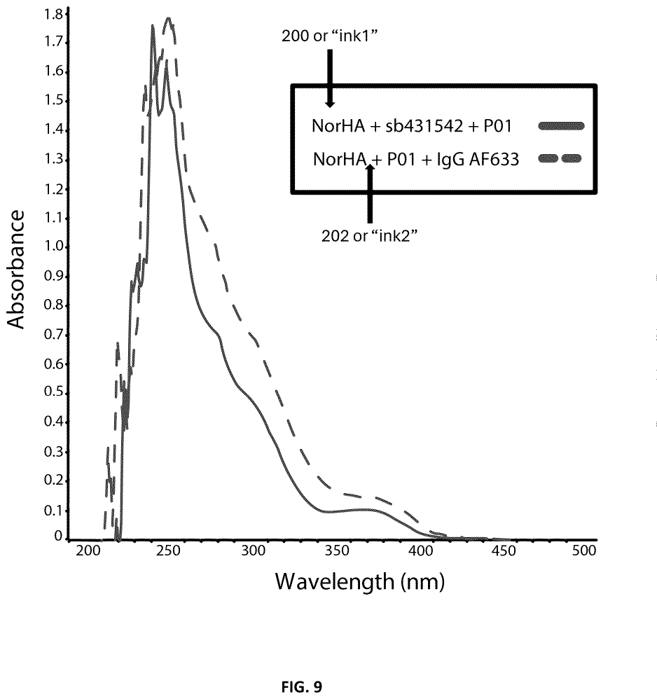

The combination of a small molecule drug and peptide on a single carrier, in one embodiment this can include SB431542 and the P01 peptide, is designated as carrier network 200 or “ink1”. The combination of a large molecule drug and peptide on a single carrier, in one embodiment this can include IgG and the P01 peptide, is designated as carrier network 202 or “ink2”. Finally, the combination of both small and large molecule drugs and a peptide on a single carrier, in one embodiment this can include SB431541, IgG, and the P01 peptide, is designated as carrier network 204 or “ink3”. The successful conjugation of “ink1” and “ink2” are verified by UV-VIS in FIG. 9.

### Blending of Pluronic F127 and Synthesized Carriers

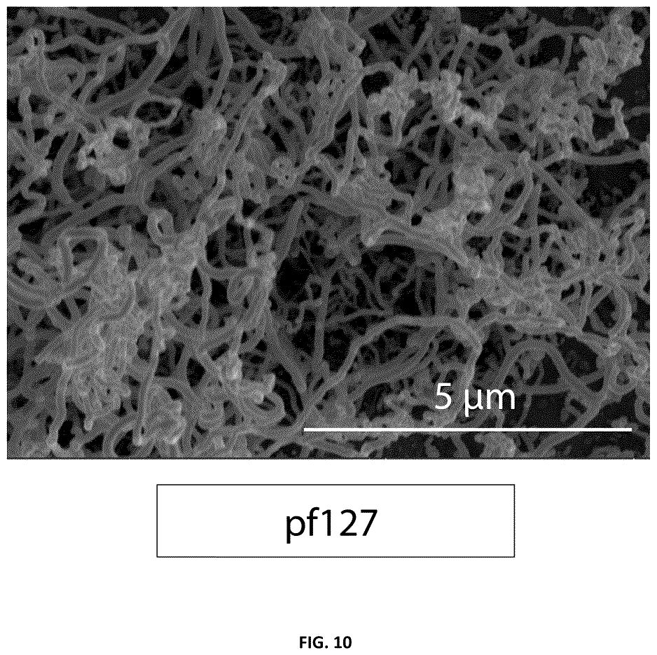

Method 100 continues with blending Pluronic F127 with ink1, ink2, or ink3 in process 150. Process 150 is initiated by preparing a mixture of Pluronic F127 (pf127) 20 wt. % by adding 2 g of Pluronic F127 into 10 mL of sterile distilled water at 50° C. for 1 hour, then returning the mixture to room temperature for 12 hours and sterile filtering through a 0.2 micron syringe filter. The mixture can be stored at 4° C. Elevation to 37° C. results in the formation of a network due to interactions between its hydrophilic and hydrophobic domains, which is shown in a scanning electron micrograph in FIG. 10.

Finally, a solution is prepared with the hydrogel and the synthesized carrier (ink1 or ink2 or ink3), the solution having a composition of 90% by volume 20 wt. % pf127 and 10% by volume of 380 nM synthesized carrier.

FIGS. 3A-3D illustrate the combination of the synthesized carrier with pf127 hydrogel, which includes a hydrophilic domain 230 (represented by curved lines) and a hydrophobic domain 240 (represented by small dark-filled circles). The resulting mixture can be loaded into a syringe as shown in FIG. 3D.

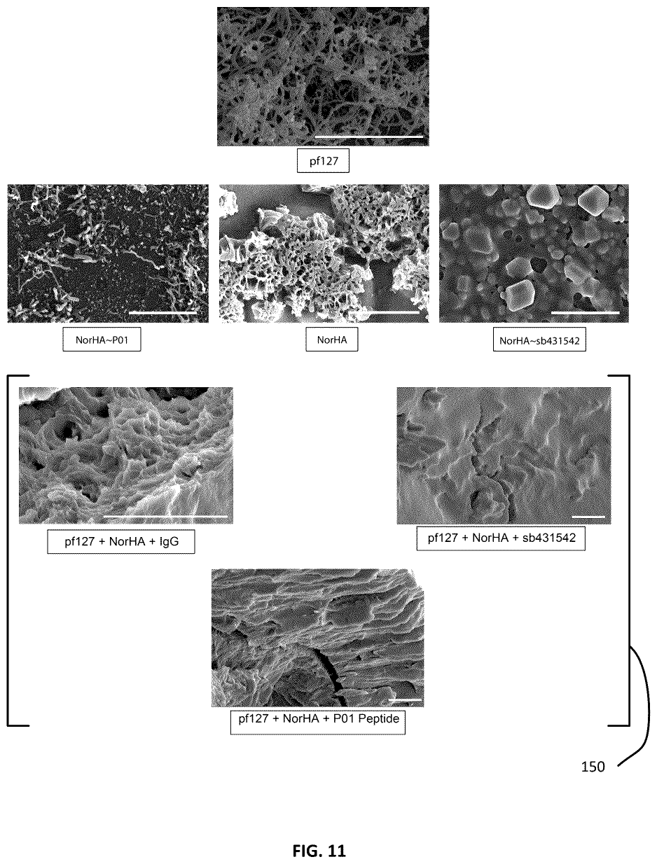

Scanning electron micrographs of the pf127 hydrogel and different forms of the synthesized carriers, both individually and after mixing using process 150, are shown in FIG. 11.

Note that the processes in method 100 are shown in a particular order for ease of description. However, one or more of the processes may be performed in a different order or may not be performed at all (and thus be optional), in accordance with some embodiments. Note also that the processes discussed above for method 100 contemplate alternative approaches and reagent substitutions.

Polysaccharides including chitosan, cellulose, and glycogen are interchangeable polymers that may be used instead of hyaluronic acid. Additionally, polyethylene glycol and its derivative polyethylene oxide also may be used for norbornene functionalization and use as a carrier molecule for therapeutic delivery in place of hyaluronic acid. Furthermore, other classes of bicyclo[2,1,1] aromatic “ene” derivatives may be used in place of 5-Norbornene 2-methylamine. These include but are not limited to Bicyclo[2.1.1]oct-2-ene and Bicyclo[2.1.1]hex-2-ene in both the amine and acid form.

For example, in the process 120 of bioconjugation of SB431542 to Norbornene-Hyaluronic Acid, any primary amine or amide bioactive domain can be functionalized through carbidimide chemical bioconjugation to the carboxylic acid domain on NorHA. An example of a small molecule is serotonin. Numerous variations on method 100 and the techniques described herein will be apparent in light of this disclosure.

For example, in the process 125 of bioconjugation of IgG to Norbornene-Hyaluronic Acid, any primary amine or amide bioactive domain can be functionalized through carbodiimide chemical bioconjugation to the carboxylic acid domain on NorHA. Examples of a large molecule include Neurotrophin 3 (NT-3) and Brain-Derived Neurotrophic Factor (BDNF).

Additionally, in the process of photoconjugation 140 of the P01 peptide to Norbornene-Hyaluronic Acid, any peptide containing cysteine residues can exploit the photoconjugation bioactive site via Michael-addition like reaction to the norbornene molecule. Examples include peptides having a sequence selected from (i) GCGGGRQVGRYLTFA, (ii) GCGGRQVGRYLTFA, and (iii) GCGRQVGRYLTFA.

Further, in the process of blending 150 Pluronic F127 with ink1, ink2, or ink3, other types of thermo-responsive hydrogel carriers could be used as a substitute. For example, any thermally responsive co-polymer and tri-polymer blocks can be used, such as polyethylene glycol/polylactic acid-co-glycolic acid (PEG/PLGA) Regel, polyoxyethylene, polyoxypropylene (PPO), pluronics, poloxamers, and Tetronics, triple blocks of copolymers polyoxyethylene-PPO-polyoxyethylene and PEG-PLA, and (polylactic acid)-PEG.

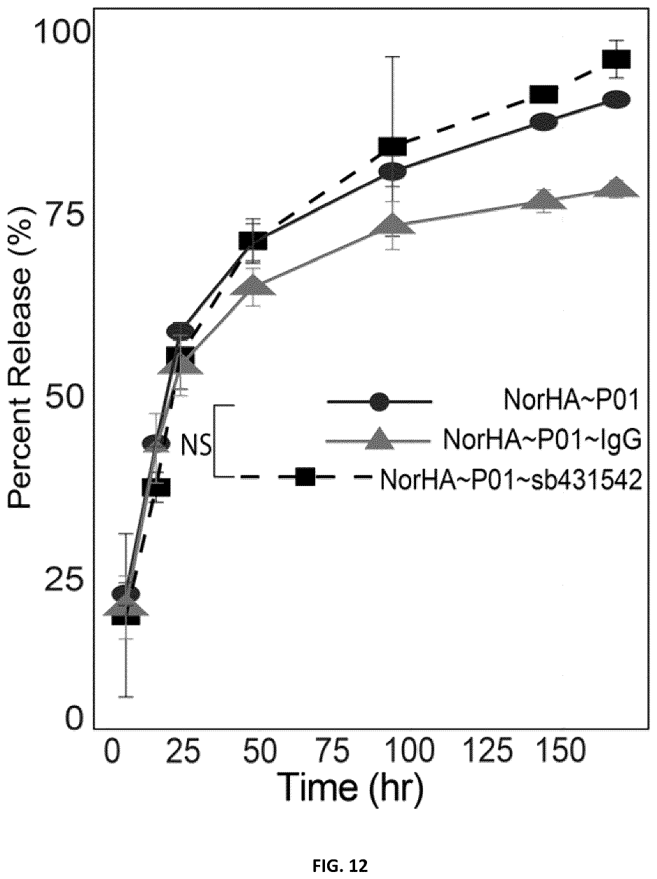

Characterization of the blended hydrogel carrier includes tracking the release rate of different carrier compositions. To track diffusion rate over time, P01 peptide was conjugated as previously described to NorHA and blended into the pf127 hydrogel. 200 μL of 1×PBS was added on top of the gels prior to placing the well plate on a rocker at 37° C. Samples were taken across several time points 6, 16, 24, 48, 96, 144, and 168 h, and then replaced with 75 μL of fresh PBS to maintain a constant volume of fluid on the gels. Nanocarrier cargo configurations were altered and characterized to evaluate their effect on release kinetics. In addition to P01 peptide, IgG and SB431542 were conjugated to NorHA as previously described. Prior to starting the release study, calibration curves for both NorHA-P01 peptide and NorHA-IgG-P01 were performed and a nonlinear least squares Michaelis-Menten fit was applied to determine diffusion of each condition in weight. Each of the synthesized configurations were screened on the Nanodrop to determine the raw amount of micrograms added into the system. Each formulation was blended 1:10 in pf127 and added to a 96-well plate in triplicate. Blended hydrogels were left to polymerize at 37° C. for 10 min. 75 μl of 1×PBS was added on top of each gel and placed on a rocker. At each time-point, all 75 μl was removed and added to a 96-well transparent plate to measure tyrosine absorbance at 280 nm in both IgG and P01 peptide. Blank pf127 was used as a background control to isolate the absolute absorbance signal of tyrosine residue present in all configurations (FIG. 12).

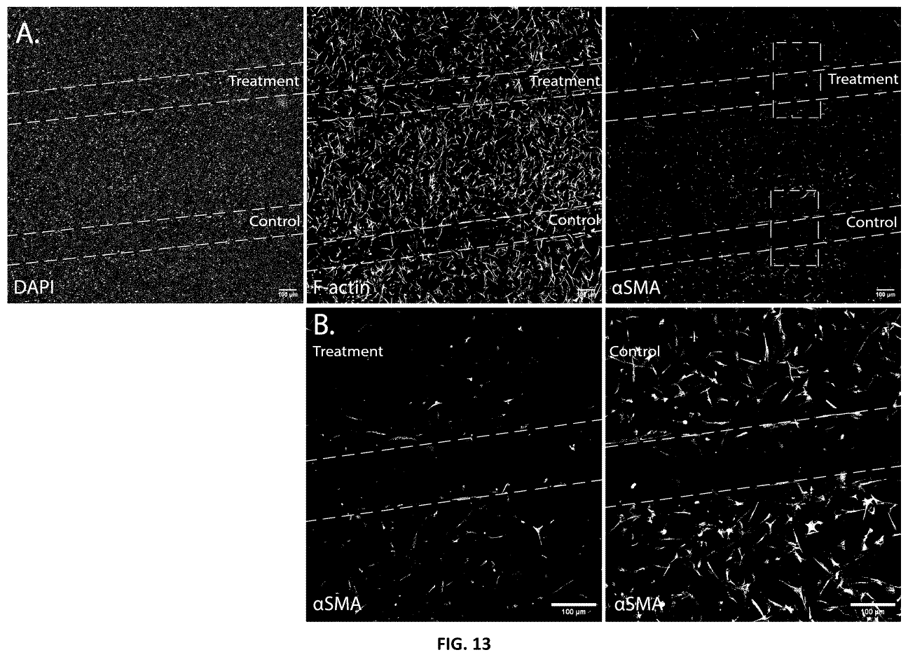

To evaluate the anti-fibrotic effects of conjugating TGF-beta inhibitor, SB431542, within the hydrogel system, P9 normal human dermal fibroblasts (hDFN) were cultured in vitro to monitor αSMA expression in a collagen hydrogel. hDFNs were seeded into a type-1 collagen hydrogel polymerized within the PDMS chamber in the presence of 300-μm diameter needles. After polymerization of the gel, the needles were pulled to create two parallel channels separated by approximately 400-μm. One channel was perfused with pf127 blended NorHA and the opposite side received pf127 blended NorHA conjugated to with P01 peptide and SB431542 inhibitor (ink1). The model was cultured in DMEM/F12 cell culture media and incubated for 3 days at 37° C. Gels were then fixed in 4% paraformaldehyde and stained for DAPI, F-actin, and αSMA. Each condition was run at n=3. αSMA fluorescence intensity was quantified in ImageJ using auto thresholding, “Triangle”, which was most representative of the source image. A single threshold was required for each image because fibroblasts adjacent to both control and treatment conditions were observed within a single frame. The sampling area for each measurement was determined by splitting the field of view in half for each gel, taking the area around the channel to measure total αSMA positive cells (FIG. 13).

### EXAMPLE USES

Carrier compositions of the present disclosure can be provided in an aqueous solution at room temperature that is ready for injection into a subject, where the solution contains hydrophobic domains that are dissociated in solution. Upon injection to a subject having a body temperature from 34-40° C., the hydrophobic domains bind together due to van der Waals forces, forming a hydrogel in the subject. The subject can be a human or an animal. For example, non-human mammals like rats provide a model for studying the effects of the injectable biomaterial on axonal regeneration after spinal cord injury. The response to injury in the rodent spinal cord is well-characterized and resembles that of humans. Therefore, the results are applicable to humans.

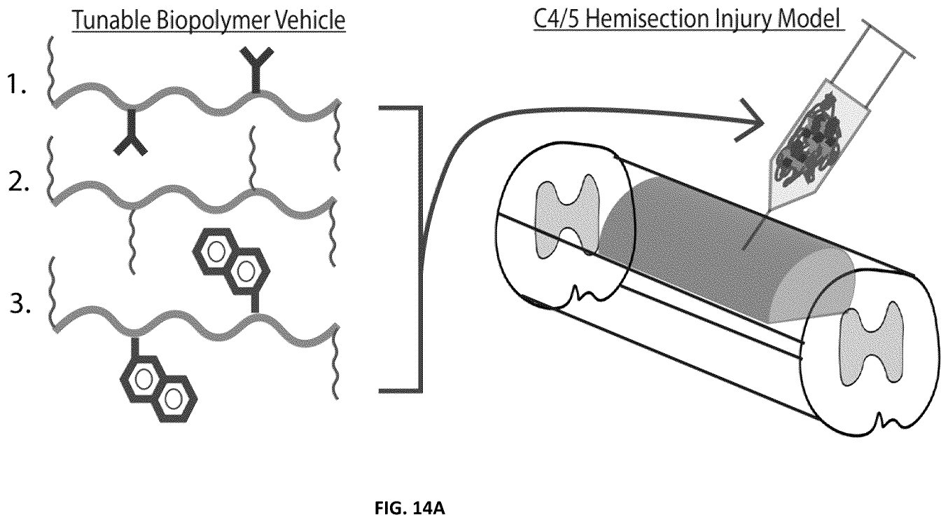

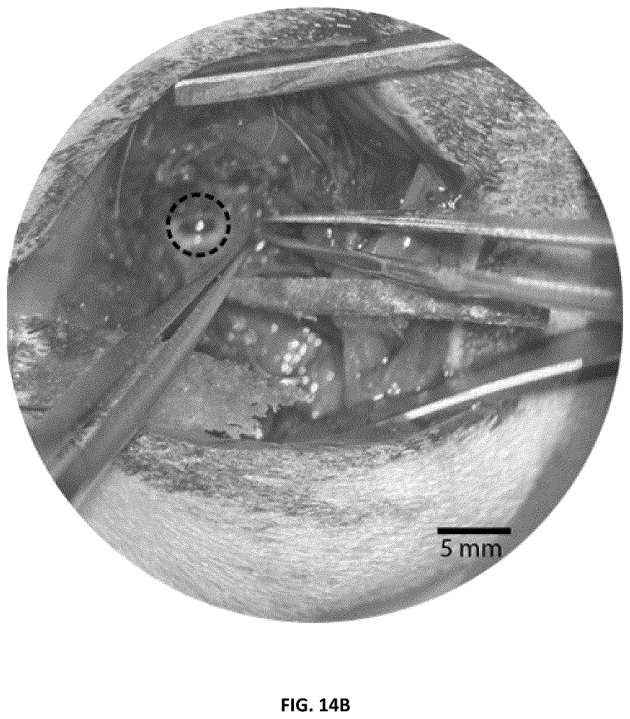

In use, the carrier composition can be injected into a subject, such as injecting within hemi-section, transection, or contusion spinal cord injury model. In one example embodiment, the composition can be delivered to a rat model of spinal cord injury by the following method: Sprague-Dawley rats (weighing approximately 200-300 g) are anesthetized with 5% of isoflurane until unconscious and then the concentration of anesthesia can be reduced to 3% during surgery. A laminectomy at the third/fourth cervical vertebrae can be performed. A C4/C5 hemisection is made at the dorsal side of the cord and the hydrogel can be delivered into the cavity by pipette. A volume of 10 μL can be inserted into the cavity and allowed to set for one minute prior to suturing the dura (shown in FIG. 14).

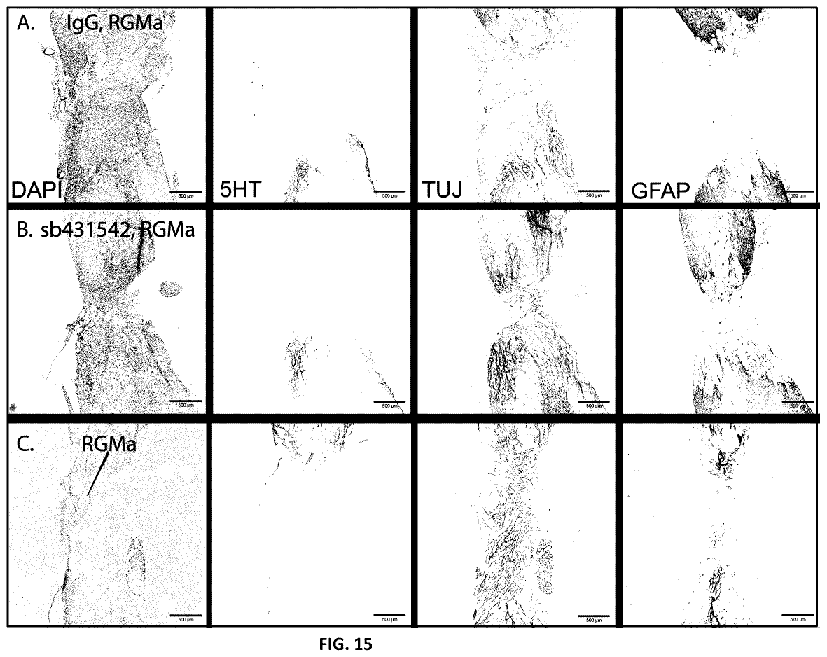

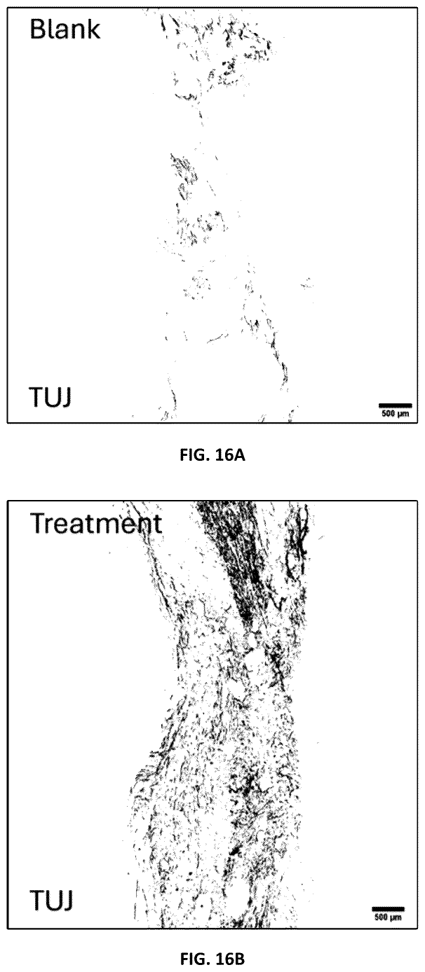

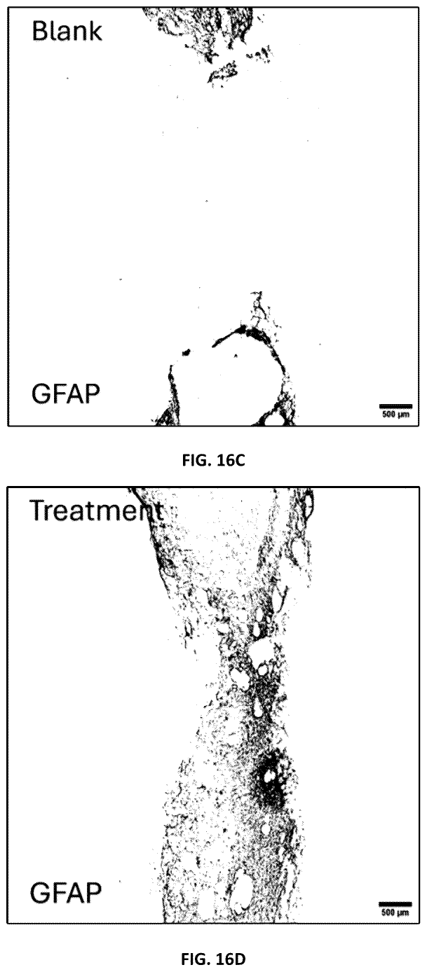

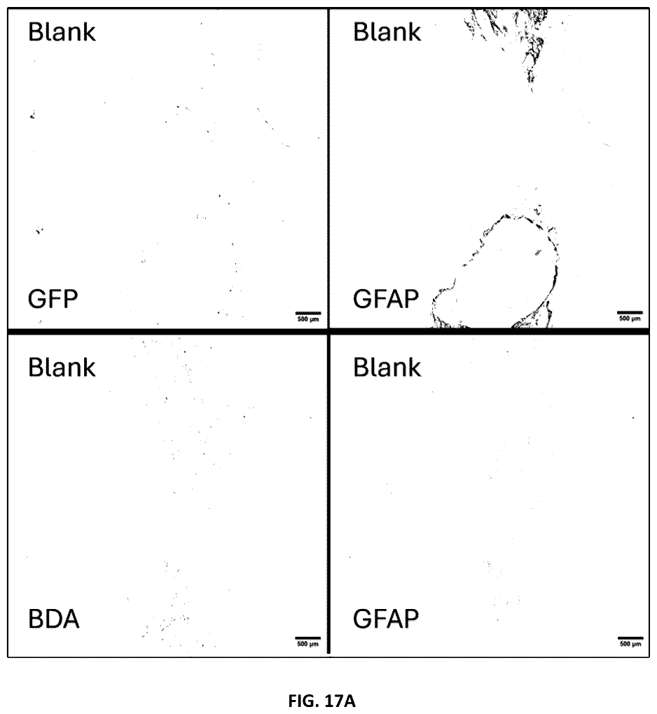

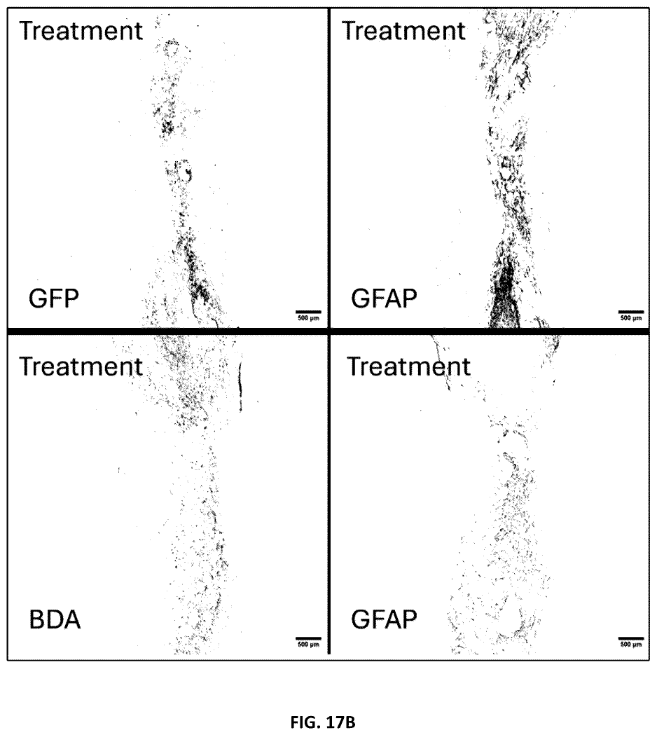

In another example embodiment, the composition can be delivered to various rat models of spinal cord injury including transection, hemisection, and contusion models. All animal experiments adhere to the guidelines of the relevant Institute of Animal Care and Use Committee. In hemisection injuries, for example, Sprague-Dawley rats (weighing approximately 200-300 g) are anesthetized with intraperitoneal injection of a mixture of ketamine/xylazine/acepromazine and a laminectomy at any position of the spinal cord (Cervical, Thoracic, Lumbar, Sacral) vertebrae is performed. Either transection or hemisection injuries are induced at the dorsal side of the cord to any area of the spinal cord (Cervical, Thoracic, Lumbar, Sacral) and the hydrogel is delivered into the cavity by pipette. A volume of 10 microLiters (μL) is inserted into the cavity and allowed to set for one minute prior to suturing the dura (Shown in FIG. 14). Evaluation of efficacy is assessed through immunocytochemical staining of key markers indicative of host neural tissue regeneration. These include Tuj (beta-tubulin III), GFAP (astrocytes), and 5-HT (motor-axons). Proof of concept studies performed in a cervical-level 5 hemisection model in Sprague-Dawley rats two-weeks post injection demonstrated different level of regeneration across three different conditions (FIG. 15). Additionally, 8-week studies using the same model compared treatment (pf127~NorHA~P01~SB431542) and control (pf127~NorHA) conditions (FIG. 16). Finally, axon tracing is performed to evaluate connectivity rostral-caudally in host tissue across different axon tracts. Injected at 5.5 weeks into the brain, BDA was used to trace reticularmotor tracts and GFP was used to trace rubrospinal tracts. Treatment conditions exhibited significant host tissue regeneration in comparison to the blank condition (FIG. 17).

### FURTHER EXAMPLE EMBODIMENTS

The following examples pertain to further embodiments, from which numerous permutations and configurations will be apparent.

Example 1 is a carrier composition comprising an amphiphilic tri-block copolymer surfactant in an amount from 10-25 wt. %; a non-sulfonated polysaccharide modified with 5-Norbornene 2-methylamine, present in an amount from 0.1-1.0 wt. %; a small molecule protein inhibitor in an amount from 0.1-1.0 wt. %; and a peptide having a sequence selected from (i) GCGGGRQVGRYLTFA, (ii) GCGGRQVGRYLTFA, and (iii) GCGRQVGRYLTFA, the peptide in an amount from 0.1-1.0 wt. %; where, at room temperature, the carrier composition comprises a polymer network in solution, and upon injection to a subject having a temperature from 34-40° C., the polymer network of the carrier composition is configured to change to a hydrogel.

Example 2 includes the carrier composition of Example 1, where the non-sulfonated polysaccharide comprises glucosaminoglycan.

Example 3 includes the carrier composition of any one of Examples 1-2, where at least 20% of subunit groups of the non-sulfonated polysaccharide are functionalized by the 5-norbornene 2-methylamine.

Example 4 includes the carrier composition of any one of Examples 1-3, where the amphiphilic tri-block copolymer surfactant comprises Pluronic F127.

Example 5 includes the carrier composition of any one of Examples 1-4, where the amphiphilic tri-block copolymer surfactant is present in an amount from 15-22 wt. %.

Example 6 includes the carrier composition of Examples 5 and further comprises immunoglobulin G present from 0.01 to 0.1 wt. %.

Example 7 includes the carrier composition of any one of the foregoing Examples, where the non-sulfonated polysaccharide that is chemically modified is present in an amount from 0.1-0.3 wt. %.

Example 8 includes the carrier composition of any one of the foregoing Examples, where the small molecule protein inhibitor is present in an amount from 0.1-0.3 wt. %.

Example 9 includes the carrier composition of any one of the foregoing Examples, where the peptide is present in an amount from 0.1-0.3 wt. %.

Example 10 includes the carrier composition of any one of the foregoing Examples, where the small molecule protein inhibitor comprises SB431542.

Example 11 includes the carrier composition of any one of the foregoing Examples, where the non-sulfonated polysaccharide that is chemically modified comprises norbornene-hyaluronic acid.

Example 12 includes the carrier composition of any one of the foregoing Examples and further comprises a small-molecule compound in the polymer network.

Example 13 includes the carrier composition of Example 12, wherein the small-molecule compound comprises serotonin.

Example 14 is a carrier composition comprising a thermally-responsive hydrogel carrier in an amount from 10-25 wt. %; a non-sulfonated polysaccharide that is functionalized with 5-Norbornene 2-methylamine, present in an amount from 0.1-1.0 wt. %; a small-molecule TGF-β inhibitor in an amount from 0.1-1.0 wt %; and a neuronal growth peptide having an amino acid sequence selected from (i) GCGGGRQVGRYLTFA, (ii) GCGGRQVGRYLTFA, and (iii) GCGRQVGRYLTFA, the neuronal growth peptide present in an amount from 0.1-1.0 wt. %, where, at room temperature, the carrier composition comprises a polymer network in solution, and upon injection to a subject having a temperature from 34-40° C., the polymer network of the carrier composition is configured to change to a hydrogel.

Example 15 includes the carrier composition of Example 14, where the non-sulfonated polysaccharide comprises glucosaminoglycan.

Example 16 includes the carrier composition of any one of Examples 14-15, where at least 20% of subunit groups of the non-sulfonated polysaccharide are functionalized by the 5-norbornene 2-methylamine.

Example 17 includes the carrier composition of any one of Examples 14-16, where the thermally responsive hydrogel carrier is present in an amount from 15-22 wt. %.

Example 18 includes the carrier composition of any one of Examples 14-17, where the non-sulfonated polysaccharide that is chemically modified is present in an amount from 0.1-0.3 wt. %.

Example 19 includes the carrier composition of any one of Examples 14-18, where the small molecule TGF-β inhibitor is present in an amount from 0.1-0.3 wt. %.

Example 20 includes the carrier composition of any one of Examples 14-19, where the neuronal growth peptide is present in an amount from 0.1-0.3 wt. %.

Example 21 includes the carrier composition of any one of Examples 14-20, where the thermally responsive hydrogel carrier comprises Pluronic F127.

Example 22 includes the carrier composition of any one of Examples 14-21, where the small-molecule TGF-β inhibitor comprises SB431542.

Example 23 includes the carrier composition of any one of Examples 14-22, where the non-sulfonated polysaccharide that is chemically modified comprises norbornene-hyaluronic acid.

Example 24 includes the carrier composition of any one of Examples 14-23 and further comprises a large-molecule compound in the polymer network.

Example 25 includes the carrier composition of Example 24, where the large-molecule compound comprises at least one of Neurotrophin 3 (NT-3) and Brain-Derived Neurotrophic Factor (BDNF).

Example 26 is a method of making a thermally-responsive carrier composition, the method comprising functionalizing a non-sulfonated polysaccharide with 5-Norbornene 2-methylamine or equivalent, bioconjugating the modified non-sulfonated polysaccharide to one of (i) a TGF-β small molecule inhibitor or (ii) immunoglobulin G, photoconjugating a peptide to the carrier with the TGF-β small molecule inhibitor or to the carrier with immunoglobulin G to provide a first carrier composition, blending pluronic F127 with the first carrier composition to provide a second carrier composition, and forming an aqueous solution of a drug composition and the second carrier composition.

Example 27 includes the method of Example 26, where the non-sulfonated polysaccharide comprises glucosaminoglycan.

Example 28 includes the method of any one of Examples 26-27, where functionalizing the non-sulfonated polysaccharide includes functionalizing at least 20% of subunits groups of the non-sulfonated polysaccharide

Example 29 includes the method of any one of Examples 26-28, where the modified non-sulfonated polysaccharide is bioconjugated to the TGF-β small molecule inhibitor and the TGF-β small molecule inhibitor comprises SB431542.

Example 30 includes the method of Example 29, where the drug composition is a small-molecule drug composition.

Example 31 includes the method of Example 30, where the small-molecule drug composition comprises serotonin.

Example 32 includes the method of any one of Examples 26-31, where the modified non-sulfonated polysaccharide is bioconjugated to the immunoglobulin G.

Example 33 includes the method of Example 32, where the drug composition is a large-molecule drug composition.

Example 34 includes the method of Example 33, where the drug composition comprises at least one of Neurotrophin 3 (NT-3) and Brain-Derived Neurotrophic Factor (BDNF).

Example 35 includes the method of any one of Examples 26-34, where functionalizing is performed using norbornene hyaluronic acid.

Example 36 is a method of treating central nervous system damage, the method comprising providing the carrier composition of any one of Examples 1-25 at room temperature and injecting the carrier composition into a subject at the site of the central nervous system damage, the subject having a body temperature from 34-40° C.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future-filed applications claiming priority to this application may claim the disclosed subject matter in a different manner and generally may include any set of one or more limitations as variously disclosed or otherwise demonstrated herein.

### NOMENCLATURE

NorHA: Norbornene-Hyaluronic Acid
HA-TBA: Hyaluronic Acid-tert butyl alcohol
BOP: BOP, Benzotriazole-1-yl-oxy-tris-(dimethylamino)-phosphoniumhexafluorophosphate (“Castro's Reagent”)
DMSO: Dimethyl sulfoxide
Nor-Amide: Norbornene Amide
NaCl: Sodium Chloride
MES: (2-(N-morpholino) ethanesulfonic acid)
NHS: N-Hydroxysuccinimide
EDC: 1-Ethyl-3-(3-dimethylaminopropyl) carbodiimide
PBS: Phosphate Buffered Saline
IgG: Immunoglobulin G
SB431542: TGF-beta small molecule inhibitor
MWCO: Molecular Weight Cut Off
P01: Peptide with Amino Acid Sequence GCGGGRQVGRYLTFA
LAP: Lithium phenyl-2,4,6-trimethylbenzoylphosphinate
Pf127: Pluronic F127
Ink1: Synthesized Carrier Containing Small Molecule and Peptide
Ink2: Synthesized Carrier Containing Large Molecule and Peptide
Ink3: Synthesized Carrier Containing Small and Large Molecules and Peptide

1. A carrier composition comprising:
an amphiphilic tri-block copolymer surfactant in an amount from 10-25 wt. %;
a non-sulfonated polysaccharide with at least 20% of subunit groups functionalized by 5-Norbornene 2-methylamine, present in an amount from 0.1-1.0 wt. %;
a small molecule protein inhibitor in an amount from 0.1-1.0 wt. %; and
a peptide having a sequence containing either an amino or carboxy terminus GCG motifs in an amount from 0.1 to 1.0 wt. %;
wherein, at room temperature, the carrier composition comprises a polymer network in solution, and upon injection to a subject having a temperature from 34-40° C., the polymer network of the carrier composition is configured to change to a hydrogel.

2. The carrier composition of claim 1, wherein the non-sulfonated polysaccharide comprises glucosaminoglycan.

3. The carrier composition of any one of claim 1, wherein the amphiphilic tri-block copolymer surfactant comprises Pluronic F127 present in an amount from 15-22 wt. %.

4. The carrier composition of claim 1, wherein the peptide is present in an amount from 0.1-0.3 wt. %.

5. The carrier composition of claim 1, wherein the small molecule protein inhibitor comprises SB431542.

6. The carrier composition of claim 1, wherein peptide comprises a sequence selected from (i) GCGGGRQVGRYLTFA, (ii) GCGGRQVGRYLTFA, and (iii) GCGRQVGRYLTFA.

7. The carrier composition of claim 1, wherein the non-sulfonated polysaccharide comprises norbornene-hyaluronic acid.

8. The carrier composition of claim 1, further comprising a small-molecule compound in the polymer network.

9. The carrier composition of claim 1, further comprising a large-molecule compound in the polymer network.

10. A carrier composition comprising:
an amphiphilic tri-block copolymer surfactant in an amount from 10-25 wt. %;
a non-sulfonated polysaccharide with at least 20% of subunit groups functionalized by 5-Norbornene 2-methylamine, present in an amount from 0.1-1.0 wt. %;
an immunoglobulin G molecule in an amount from 0.01-0.1 wt. %; and
a peptide sequence containing either an amino or carboxy terminus GCG motifs in an amount from 0.1 to 1.0 wt. %;
wherein, at room temperature, the carrier composition comprises a polymer network in solution, and upon injection to a subject having a temperature from 34-40° C., the polymer network of the carrier composition is configured to change to a hydrogel.

11. The carrier composition of claim 10, wherein the non-sulfonated polysaccharide comprises a glucosaminoglycan.

12. The carrier composition of claim 11, wherein the amphiphilic tri-block copolymer surfactant comprises Pluronic F127 in an amount from 15-22 wt. %.

13. The carrier composition of claim 10, wherein the immunoglobulin G is a monoclonal antibody raised against an inflammatory cytokine, an axonal repulsive guidance cue, or another ligand associated with secondary injury.

14. The carrier composition of claim 10, wherein the peptide has a sequence selected from (i) GCGGGRQVGRYLTFA, (ii) GCGGRQVGRYLTFA, and (iii) GCGRQVGRYLTFA.

15. The carrier composition of claim 10, wherein the non-sulfonated polysaccharide comprises norbornene-hyaluronic acid.

16. The carrier composition of claim 10, wherein the non-sulfonated polysaccharide is functionalized with both (i) an immunoglobulin G molecule in an amount from 0.01-0.1 wt. % and (ii) a small molecule protein inhibitor in an amount from 0.1-1.0 wt. %.

17. A method of making a thermally responsive carrier composition, the method comprising:
functionalizing a non-sulfonated polysaccharide with 5-Norbornene 2-methylamine or equivalent on at least 20% of its subunit groups;
bioconjugating the modified non-sulfonated polysaccharide to (i) a TGF-β small molecule inhibitor and/or (ii) immunoglobulin G;
photoconjugating a peptide to the non-sulfonated polysaccharide to provide a first carrier composition;
blending pluronic F127 with the first carrier composition to provide a second carrier composition; and
forming an aqueous solution of a drug composition and the second carrier composition.

18. The method of claim 17, wherein the non-sulfonated polysaccharide comprises a glucosaminoglycan.

19. The method of claim 17, wherein the modified non-sulfonated polysaccharide is bioconjugated to the TGF-β small molecule inhibitor and wherein the TGF-β small molecule inhibitor comprises SB431542.

20. A method of treating central nervous system damage in a live subject, the method comprising:
providing the carrier composition of claim 1 at room temperature; and
injecting the carrier composition into the live subject at a site of the central nervous system damage, the subject having a body temperature from 34-40° C.


SEQUENCE LISTING (3 sequences)

SEQ ID NO: 1 - protein, 15 aa, synthetic construct
GCGGGRQVGRYLTFA

SEQ ID NO: 2 - protein, 14 aa, synthetic construct
GCGGRQVGRYLTFA

SEQ ID NO: 3 - protein, 13 aa, synthetic construct
GCGRQVGRYLTFA